(19) United States
(12) Patent Application Publication
(10) Pub. No.: US 2008/0115549 A1
(43) Pub. Date: May 22, 2008

(54) NOVEL AQUEOUS COMPOSITION AND USE OF THE SAME

(75) Inventors: Shoji Yamashita, Ibaraki (JP); Mamoru Ando, Ibaraki (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Institute for State Physics of Natural Materials, Ibaraki (JP)

(21) Appl. No.: 11/931,737

(22) Filed: Oct. 31, 2007

Related U.S. Application Data
(63) Continuation of application No. 11/560,549, filed on Nov. 16, 2006, now abandoned.
Application No. 11/560,549 is a continuation of application No. 11/157,912, filed on Jun. 22, 2005, now abandoned.
Application No. 11/157,912 is a continuation of application No. 10/752,100, filed on Jan. 7, 2004, now abandoned.
Application No. 10/752,100 is a continuation of application No. 10/168,865, filed on Jun. 26, 2002, now abandoned.

(30) Foreign Application Priority Data
Dec. 26, 1999 (JP) 11-377077

(51) Int. Cl. C09K 15/02; C09K 3/00; C05D 9/00; C12N 5/00

(52) U.S. Cl. 71/31; 252/380; 252/397; 252/387; 435/325

(57) ABSTRACT

An aqueous composition containing a single compound of a ferrosoferric salt as the active ingredient. The composition has many uses, including use as a rust preventative.

### BACKGROUND OF THE INVENTION

1. Field of the Invention

The present invention relates to an aqueous composition of an iron salt of a single compound comprising a ferrosoferric salt and uses thereof.

2. Background of the Invention

A ferrosoferric salt of formula (A:)

Fe(II)ₘFe(III)ₙY^{-z}_{(2m+3n)/Z},

wherein each of m and n is a positive integer, Y is a counter anion of a ferrosoferric cation and Z is the total anionic charge of anion Y, is a known compound as disclosed in Japanese Patent Publication of 362023/1992. The publication also describes the use of the compound (See the first paragraph of Japanese Patent Publication of 362023/1992) which detoxifies inorganic chromium VI by its deoxability.

Japanese Patent Publication of 190226/1984 describes a ferrosoferric salt which contains sodium chloride (See page 2 line 13 of the left column), Example 1 of the publication shows that the ferrosoferric salt is known to possess rust inhibition ability, and Example 2 shows the ability of the salt to eliminate salt hindrance of plants. Example 3 describes the ability of the ferrosoferric salt to improve upon the problem of crop development which occurs as a result of the continuous planting of the same crop in the same area of soil. Example 4 describes the ability of the salt to stably store tissue of a living body. Example 5 describes the ability of the salt to revive tissues of plants. Example 6 describes the ability of the salt to promote inorganic synthesis of components of a living body. Example 7 describes the aseptic ability of the salt and its ability to prevent the growth of mold. Example 8 describes the anti-virus ability of the salt and Example 9 describes the anti-tumor property of the salt.

The density of aqueous ferrosoferric salt solutions disclosed in Japanese Patent Publication 190226/1984 are, however, limited such as 2.5×10⁻³ g/ml in Ex. 1, 10⁻¹³ g/ml in Ex. 2, 10⁻¹² g/ml in Ex. 3, 10⁻⁶/ml in Ex. 4, 10⁻⁷ g/ml in Ex. 5 and 10⁻⁶ g/ml in Examples 6-9. The activity of a given aqueous salt solution depends on the density of the ferrosoferric salt in relation to a specific use of the composition. The known aqueous compositions present complicated problems such as the adjustment of the density of a given ferrosoferric salt solution for a given specific application. Accordingly, a desirable objective for the preparation of ferrosoferric salt solutions is to prepare a solution whose activity in a particular use is not density dependent.

### SUMMARY OF THE INVENTION

The present inventors have conducted extensive research with the objective to provide an aqueous composition comprising a ferrosoferric salt which does not present a density dependency factor for the ferrosoferric salt in a given application. It has now been found that an aqueous composition comprising a ferrosoferric salt of formula (A):

Fe(II)ₘFe(III)ₙY^{-Z}_{(2m+3n)/Z},

wherein each of m and n is a positive integer, Y is a counter anion of a ferrous/ferric cation and Z is the total anionic charge of anion Y, is effective as a rust preventive agent for metals, an agent which eliminates salt hindrance of plants, an agent which improves upon the obstacle presented by the continuous planting of the same crop in the same area of soil, an agent which permits stable storage of tissues of bioorganisms, an agent which permits stable storage of plant tissues, a non-biologically synthetic agent for bioorganism components, an antiseptic agent for plants, an anti-mold agent, an anti-virus agent and an anti-tumour agent, and which effectiveness is independent on the density of aqueous solutions of the ferrosoferric salt.

The aqueous composition on the present invention is different from that of Japanese patent publication of 190226/1984 in that its effectiveness is not dependent on the density of the ferrosoferric salt.

### DESCRIPTION OF THE PREFERRED EMBODIMENTS

The present invention relates to an aqueous composition comprising a ferrosoferric salt of formula (A):

Fe(II)ₘFe(III)ₙY^{-Z}_{(2m+3n)/Z},

wherein each of m and n is a positive integer, Y is a counter anion of a ferrosoferric cation and Z is the total anionic charge of anion Y, and its use as a rust preventive agent for metals. The aqueous composition is prepared by dissolving a ferrosoferric salt of formula (A): in water and further diluting the solution suitably with water.

Distilled water, hard water and soft water are examples of water that can be used. Distilled water is preferred because it provides a composition of greater stability although any aqueous solution can be used to prepare the composition.

The pH value of the aqueous composition is not limited as long as the ferrosoferric salt of the solution is converted to an insoluble iron salt such as Fe(OH)₂ and ranges from 3.0 to 9.0, preferably from 5.0 to 7.5, more preferably 5.5 to 6.0.

Now, the preparation of the aqueous composition of the present invention will be described.

The aqueous composition of the present invention is prepared by dissolving a ferrosoferric salt of formula (A) in water by the addition of an aqueous solution to a ferrosoferric salt or by the addition of a ferrosoferric salt to an aqueous solution and further diluting the solution with an aqueous material such as distilled water.

The usual manner of dissolving the salt is by stirring. The solution can be heated in the event dissolution does not easily occur and depending on the kind of the aqueous solvent employed, although the ferrosoferric salt of the present invention is soluble in water. Filtration can be preferably conducted to remove precipitated matter from the aqueous solution which is derived from impurities in the solvent.

An aqueous composition comprising a ferrosoferric salt falls within the scope of the claims of the present invention, as long as the ferrosoferric salt dissolves in the solution, since the effectiveness of the composition does not depend on the density of the ferrosoferric salt. The density of the aqueous ferrosoferric salt solution preferably is 10⁻¹ to 10⁻⁷ g/ml, more preferably 10⁻³ to 10⁻⁵ g/ml.

Next, the ferrosoferric salt of formula (A): as an active ingredient of the aqueous composition of the present invention will be described.

Suitable examples of anion Y include inorganic anions such as chloride, sulfate and nitrate or organic anions such as formate, acetate, oxalate, succinate, a malate ion, a tartrate ion, a fumarate ion, and a citrate ion.

The ratio of m/n in formula A is determined by the species of bivalent metal salt used in the first method or by the prescribed dilution density in the second method. Particularly, preferred ratios of m/n are 2/3, 1/1, 3/2, 2/1 and 7/3.

The process for the preparation of the ferrosoferric salts is described in detail with reference to the preferred embodiments. The ferrosoferric salt is prepared by the first and the second method.

Suitable trivalent iron salts for the preparation of the ferrosoferric salt include known iron salts such as FeCl₃, Fe₂(SO₄)₃, Fe(NO₃)₃ and the solvates thereof.

Suitable bivalent metal salts for the preparation of the ferrosoferric salt include, for example, CaCl₂, MgCl₂, ZnCl₂₁ MgSO₄, Ca(NO₃)₂, Mg(NO₃)₂ and Zn(NO₃)₂.

Suitable organic acids for use in the preparation of the salt include, for example, formic acid, acetic acid, oxalic acid, succinic acid, malic acid, tartaric acid, fumaric acid and citric acid.

The ferrosoferric salt of the present invention may be prepared by the dissolution of a trivalent iron salt in an aqueous solution containing a trivalent iron salt and a bivalent metal salt in specific concentrations and then concentration of the solution obtained (Method 1).

The ferrosoferric salt of the present invention may also be prepared by the dissolution of a trivalent iron salt in an aqueous solution containing a trivalent iron salt and an organic acid ranging in specific concentrations and of specific electric conductivity, and then concentration of the solution obtained (Method 2).

Further as to Method 1, the first step of the method is to prepare an aqueous solution comprising a trivalent iron salt and an organic acid each in prescribed concentration ranges such as the same equivalent molar ratio. After the aqueous solution is diluted with distilled water to give a second aqueous solution containing a trivalent iron salt and an organic acid in specified concentration (˜10⁻¹⁰ mM), a trivalent iron salt is then dissolved in the second aqueous solution. The solution thus obtained is concentrated at 100° C. to give the desired ferrosoferric salt.

Further as to Method 2, the first step of the method is to prepare an aqueous solution comprising a trivalent iron salt and an organic acid each in a prescribed ratio (for example a trivalent iron salt/an organic acid=½ (per mole)). The aqueous solution is subsequently diluted ten times with distilled water to give a series of diluted aqueous solutions (concentration: 10⁻⁴˜10⁻²⁰ mM). The electric conductivity of each diluted solution is measured, and solutions having an electrical conductivity of more than 3 μs/cm as a maximum electrical conductivity are selected. A trivalent iron salt is added to a selected solution or a mixture of one or more selected solutions, and the solution thus obtained is concentrated at 100° C. to give the desired ferrosoferric salt.

The results of the existing state of the crystalline powder of the ferrosoferric salt measured by ion chromatography and X-ray crystal structure analysis support the fact that the ferrosoferric salt exists not as a mixture but as a single compound.

The aqueous composition comprising a ferrosoferric salt prepared by Method 1 or Method 2, may be used as a rust preventive agent for metals, as an agent which eliminates salt hindrance, as an improvement agent for soil for removing the obstacles presented by the continuous planting of a crop, as an agent which permits stable storage of tissues of bioorganisms, as an agent which permits stable storage of plant tissues, as a non-biologically synthetic agent for bioorganism components, as an antiseptic agent for plants, as an anti-mold agent and as an anti-virus agent.

The tests described below demonstrate the various properties of the ferrosoferric salt of the invention as described immediately above.

### Test 1: Antitrust Ability

After an iron plate (0.2 cm×5 cm×5 cm) was washed with aqueous diluted HCl and distilled water and then dried, the iron plate was saturated with the aqueous composition of Ex. 1, and allowed to stand for 30 minutes at 80° C. As a reference, the iron plate was soaked in distilled water, and allowed to stand for 30 minutes at 80° C. Both iron plates were left under HCl gas stream and the surface of the iron plate was observed. The reference iron plate exhibited significant rust development after 1 hour from the beginning of the test, whereas the iron plate treated with the aqueous composition of Ex. 1 exhibited no rust even after 6 days from the start of the test. Because the iron plate treated with the aqueous composition of the present invention exhibited no rusting even under the severe corrosive HCl atmosphere, the aqueous composition of the present invention showed a significant ability to preventing rusting of metals.

### Test 2: Elimination for Salt Hindrance

The addition of the aqueous composition of the present invention to natural sea water afforded a test solution whose concentration of a ferrosoferric salt is around 10⁻⁴ g/ml. Iron powder, magnesium powder and copper powder were added to the test solution and untreated sea water as a reference. While chlorides of all the metals were produced within a day in the reference sea water which was not treated, there were no chlorides in the test solution. The result of the test is that no salt hindrance of the test solution, and the powdered metals were stable in the test solution.

### Test 3: Improvement of Soil in View of Obstacle of Continuous Crop Growth

To soil having the difficult-to-treat obstacle of the breeding of fusarium species in cultivated land used for the growth of Japanese radishes, was added the aqueous composition of Example 3. The soil was moistened with the present aqueous composition. The radishes were planted in the soil in the ordinary manner. As a result all of the radishes grew to achieve a 240 ratio yield versus 100 ratio yield of reference untreated soil. From the above results, the aqueous composition of the present invention provides improved hindrance soil and can lead to proper plant growth.

### Test 4: Restored of Animal Tissue

Fresh muscle tissue excised from a white mouse which had just been killed was placed into a bottle and the aqueous composition of Example 2 was added thereto. The bottle was stopped enclosing the air therein and the bottle was gently kept at room temperature. A reference of untreated muscle tissue was also prepared. The muscle tissue of the reference had deteriorated within a week from the start of the test, while the muscle tissue in the aqueous composition of the present invention had not deteriorated. Microbes were not breeding in the tissue and the solution was clear and retained this state during the time the tissue sample was bottled. From the above result, the aqueous composition of the present invention permits the storage of living tissue in a stable manner.

### Test 5: Revival of a Plant Tissue

Twigs of a black pain were steeped in a sample of the aqueous composition of Ex. 3 and a sample of distilled water (reference) for 30 minutes, and the treated and untreated twigs were then placed in quartz sand in a pot. Although all the untreated twigs of the reference test withered, whereas the twigs treated with the composition of the invention had sprightly taken root. From the above results, the aqueous composition of the present invention may significantly facilitate the revival of the cut portions of plant tissue, and the growth of plants.

### Test 6: Aseptic and Prevention of Mold

Shucked clams and pieces of rice cake which had been exposed in an open system at 32° C. for 3 days had microorganisms breeding therein. To 10 ml of aqueous composition of Ex. 1 in a test tube was added a sample of the growing microorganisms together with each of 0.5 g of rice powder and peptone. The combination was allowed to stand for 3 days at 32° C. To 100 ml of distilled water was added 0.1 ml of the obtained suspension to prepare the solution for the test. Fresh shucked clams and pieces of rice cake were preserved in the test solution of a sealed bottle at room temperature. A reference test was conducted using distilled water instead of the aqueous composition as a test solution. Although microorganisms and mold formed in the reference solution of the test system, multiplication of microorganisms did not occur in the test system treated with the aqueous composition. From these results, the aqueous composition of the present invention exhibits a remarkable aseptic ability and ability to prevent mold growth.

### Test 7: Anti-Virus Ability

After the leaf of a tomato plant was inoculated with TMV as a host plant and the virus was allowed to propagate in vivo, a TMV test suspension was prepared by diluting squeezed solution from the leaf 500 times with distilled water just before the test. After the leaf of the tobacco plant had grown for a month to which was applied Carborundum™, a TMV suspension diluted twice with distilled water was applied to a half part of a test leaf with cotton. The test TMV suspension was prepared by using the aqueous composition of Ex. 1. After the leaf was dried, the remainder of the Carborundum™ was washed with water, and the leaf was allowed to grow in Koitotolon™ at 26° C. The number of spots of the leaf tested and inhibition rate of the test composition were measured.

### The result was shown in Table 1.

TABLE 1

| | Reference | Test | Inhibition rate |
|---|---|---|---|
| Number of spots (1) | 125 | 0 | |
| Number of spots (2) | 179 | 6 | |
| Number of spots (3) | 66 | 3 | |
| Average | 128.3 | 3 | 97.6% |

From the above result, the aqueous composition of the present invention exhibit anti-virus ability.

Antirusting tests for metals were conducted in various concentrations of a ferrosoferric salt of Reference Ex. 1 of the present invention and iron chloride (II, III) disclosed in Japanese Patent Publication 190226/1984 in comparison with a non-treatment test.

### Test. 8

After an iron plate (0.2 cm×5 cm×5 cm) which had been washed with aqueous diluted HCl and distilled water and dried, a prescribed test solution (200 ml) of the ferrosoferric salt of Reference Ex. 1 of the present invention, iron chloride (II, III) disclosed in Japanese Patent Publication of 190226/1984, hydrogen fluoride (1.2×10⁻⁴ g/ml) and glucose (1.0×10⁻³ g/ml) was prepared. The iron plate was saturated with the above test solution, and allowed to stand for 30 minutes at 80° C. Iron plates for the test were left under a HCl gas stream, and the surfaces of the iron plates were observed. The results are shown in Table 2.

TABLE 2

| | Concentration of | The state of | The state of |
|---|---|---|---|
| | ferrosoferric | corrosion of the | corrosion of the |
| | salt of | surface after | surface after |
| Test compound | test (g/ml) | 1 hour | 6 days |
| reference (no | 0 | + | + |
| treatment) | | | |
| Compound of | 2.5 × 10⁻¹ | − | − |
| REFERENCE | 2.5 × 10⁻³ | − | − |
| EXAMPLE 1 of the | 2.5 × 10⁻⁵ | − | − |
| present application | 2.5 × 10⁻⁷ | − | − |
| iron chloride | 2.5 × 10⁻¹ | + | + |
| (II, III) | 2.5 × 10⁻³ | − | − |
| carrying sodium | 2.5 × 10⁻⁵ | + | + |
| chloride) | 2.5 × 10⁻⁷ | + | + |

(+: corrosion; −: no corrosion)

The reference iron plate exhibited significant rusting after 1 hour from the beginning of the test, whereas the iron plate treated with aqueous composition of a ferrosoferric salt did not exhibit corrosion. Moreover, the aqueous composition of the present invention showed an antirusting effect at every concentration of the salt, and the aqueous composition comprised of iron chloride, disclosed in Japanese patent publication of 190226/1984, derived from iron chloride II, showed an antirusting effect only at certain salt concentrations.

### EXAMPLES AND REFERENCE EXAMPLES

The following examples illustrate the present invention more specifically. It should be understood that the present invention is not limited to the examples alone.

### Example 1

100 mg of the compound of Reference example 5 was dissolved in 10 L of distilled water (pH 6.5) at room temperature, and the obtained solution was diluted 100 times using the above distilled solution to yield the objective aqueous composition.

### Example 2

100 mg of the compound of Reference example 2 was dissolved in 10 L of distilled water (pH 6.0) at room temperature, and the obtained solution was diluted 1000 times using the above distilled solution to yield the objective aqueous composition.

### Example 3

100 mg of the compound of Reference example 1 was dissolved in 10 L of distilled water (pH 5.5) at room temperature, and the obtained solution was diluted 1000 times using the above distilled solution to yield the objective aqueous composition.

### Reference Examples

### Reference Example 1

In 100 ml of aqueous solution (10 mM) of CaCl₂ was dissolved 270 mg of FeCl₃.6H₂O, and the resulting solution was diluted with distilled water to yield a diluted solution (concentration of the salts: 10⁻¹⁰ mM). To 20 ml of the above diluted solution was added 1 g of crystalline FeCl₃.6H₂O and the solution was gradually concentrated in a porcelain dish over a boiling water bath. The obtained solid concentrate was dried over P₂O₅ in a desiccator.

The ratio of Fe(II) to Fe(III), measured by Mössbauer spectroscopy analysis, was 2/3. The formula of the main component of the compound thus obtained, i.e., Fe(II)₂Fe(III)₃Cl₁₃, was determined.

### Reference Example 2

In 100 ml of aqueous solution (10 mM) of ZnCl₂ was dissolved 270 mg of FeCl₃.6H₂O, and the resulting solution was diluted with distilled water to yield a diluted solution (concentration of the salts: 10⁻¹⁰ mM). To 20 ml of the above diluted solution was added 1 g of crystalline FeCl₃.6H₂O and the solution was gradually concentrated in a porcelain dish over a boiling water bath. The obtained solid concentrate was dried over P₂O₅ in a desiccator. The ratio of Fe(II) to Fe(III), measured by Mössbauer spectroscopy analysis, was 3/2. The formula of the main component of the compound thus obtained, i.e., Fe(II)₃Fe(III)₂Cl₁₂, was determined.

### Reference Example 3

In 100 ml of aqueous solution (20 mM) of NH₄CHO₂ was dissolved 270 mg of FeCl₃.6H₂O, and the resulting solution was subsequently diluted with distilled water to yield several solutions of different salt concentrations. The electric conductivity of each diluted solution was measured to determine the solution that has a maximum electrical conductivity of 14 μs/cm (concentration of the salts: 10⁻¹⁴ mM). To this solution was added 1 g of crystalline FeCl₃.6H₂O and the solution was gradually concentrated in a porcelain dish over a boiling water bath. The obtained solid concentrate was dried over P₂O₅ in a desiccator. The ratio of Fe(II) to Fe(III), measured by Mössbauer spectroscopy analysis was 7/3. The formula of the main component of the compound thus obtained, i.e., Fe(II)₇Fe(III)₃Cl₂₃, was determined.

### Reference Example 4

Fe(II)₂Fe(III)₃Cl₁₃ obtained in REFERENCE Ex. 1 was dissolved in water to prepare a diluted solution whose concentration of iron salts was 1 ppm (Solution 1). In the same manner, Fe(II)₇Fe(III)₃Cl₂₃ obtained in REFERENCE Ex. 3 was dissolved in water to prepare a diluted solution whose concentration of iron salts was 1 ppm (Solution 2). 10 ml of Solution 1 and 2.5 ml of Solution 2 were mixed, and the mixture was diluted with distilled water to prepare a diluted solution (concentration of the iron: 10⁻⁸ ppm). In 20 ml of the resulting diluted solution was dissolved 1 g of crystalline FeCl₃.6H₂O, and the solution was gradually concentrated in a porcelain dish over a boiling water bath. The obtained solid concentrate was dried over P₂O₅ in a desiccator. The ratio of Fe(II) to Fe(III), measured by Mössbauer spectroscopy analysis, was 1/1. The formula of the main component of the compound thus obtained, i.e., Fe(II)Fe(III)Cl₅, was determined.

### Reference Example 5

Fe(II)₂Fe(III)₃Cl₁₃ obtained in REFERENCE Ex. 1 was dissolved in water to prepare a diluted solution whose concentration of iron salts was 1 ppm (Solution 1). In the same manner, Fe(II)₇Fe(III)₃Cl₂₃ obtained in REFERENCE Ex. 3 was independently dissolved in water to prepare a diluted solution whose concentration of iron salts was 1 ppm (Solution 2). 3.0 ml of Solution 1 and 12.0 ml of Solution 2 were mixed, and the mixture was diluted with distilled water to prepare a diluted solution (concentration of the iron: 10⁻⁸ ppm). In 20 ml of the resulting diluted solution was dissolved 1 g of crystalline FeCl₃.6H₂O, and the solution was gradually concentrated in a porcelain dish over a boiling water bath. The obtained solid concentrate was dried over P₂O₅ in a desiccator. The ratio of Fe(II) to Fe(III), measured by Mössbauer spectroscopy analysis, was 2/1. The formula of the main component of the compound thus obtained, i.e., Fe(II)₂Fe(III)Cl₇, was determined.

### Reference Example 6

An aqueous solution comprising NH₄CHO₂ (2M), NH₂OH.HCl (1M) and HCHO (1M) was prepared, and FeCl₃.6H₂O (1 M) was added thereto, and the resulting solution was subsequently diluted with distilled water to afford solutions of various concentrations of salt. The electrical conductivity of each diluted solution was measured to determine the solution that has a maximum electrical conductivity of 3-14 μs/cm (concentration of the salts: 10⁻⁸ mM, 10⁻¹² mM and 10⁻¹⁴ mM). To 10 ml of each solution [10⁻⁸ mM (hereinafter referred to as α solution), 10⁻¹² mM (hereinafter referred to as β solution) and 10⁻¹⁴ mM (hereinafter referred to as γ solution)] was added 1 g of crystalline FeCl₃.6H₂O and each solution was gradually concentrated in a porcelain dish at a temperature less than 100° C. The obtained solid concentrates were dried in a desiccator to yield crystalline powders. The ratio of Fe(II) to Fe(III), measured by Mössbauer spectroscopy analysis, was as follows:

(1) A crystal derived from the α solution: the ratio of Fe(II) to Fe(III)=2/3, and consequently, the formula of the main component of the compound thus obtained, i.e., Fe(II)₂Fe(III)₃Cl₁₃ was determined.

(2) A crystal derived from the β solution: the ratio of Fe(II) to Fe(III)=3/2, and consequently, the formula of the main component of the compound thus obtained, i.e., Fe(II)₃Fe(III)₂Cl₁₂ was determined.

(3) A crystal derived from the γ solution: the ratio of Fe(II) to Fe(III)=7/3, and consequently, the formula of the main component of the compound thus obtained, i.e., Fe(II)₇Fe(III)₃Cl₂₃, was determined.

### INDUSTRIAL APPLICABILITY

This invention relates to an aqueous ferrosoferric salt composition which is useful as a rust preventive agent for metals and is independent of the concentration of ferrosoferric salt.

What is claimed:

1. An aqueous composition, comprising
a ferrosoferric salt of the general formula (A):
Fe(II)ₘFe(III)ₙY^{-z}_{(2m+3n)/z}
(wherein each of m and n is a positive integer, Y is a counter anion for a ferrous and a ferric cation and z is an ionic value of Y).

2. A rust preventive agent for metals comprising
an effective amount of the aqueous composition in claim 1.

3. The aqueous composition of claim 1, wherein the amount of the ferrosoferric salt of formula (A) in the aqueous solution ranges from 10⁻¹ to 10⁻⁷ g/ml.

4. The aqueous composition of claim 3, wherein the amount of the ferrosoferric salt of formula (A) in of the aqueous solution ranges from 10⁻³ to 10⁻⁵ g/ml.

5. The aqueous composition of claim 1, wherein counter anion Y is chloride, sulfate, nitrate, formate, acetate, oxalate, succinate, malate, tartrate, fumarate or citrate.

6. The aqueous composition of claim 1, wherein the ratio m/n is 2/3, 1/1, 3/2, 2/1 or 7/3.

7. The aqueous composition of claim 1, wherein the pH of the solution ranges from 3.0 to 9.0.

8. The aqueous composition of claim 7, wherein the pH of the solution ranges from 5.0 to 7.5.

9. The aqueous composition of claim 8, wherein the pH of the solution ranges from 5.5 to 6.0.

10. An aqueous ferrosoferric salt solution prepared by a process, comprising:
preparing a solution of trivalent iron salt in water containing a bivalent metal salt or organic acid which supplies counter anion for said ferrosoferric salt;
diluting the aqueous solution with water to give a second aqueous solution to a desired ferrosoferric salt concentration;
adding additional trivalent iron salt to the second aqueous solution; and then
concentrating the salt solution at a temperature under 100 C to the desired concentration of ferrosoferric salt having the formula: Fe(II)ₘFe(III)ₙY^{-z}_{(2m+3n)/z}
wherein each of m and n is a positive integer, Y is a counter anion for the ferrous and ferric cations and z is the ionic value of counter anion Y.

11. The process of claim 10, wherein the bivalent metal salt is CaCl₂, MgCl₂, ZnCl₂, MgSO₄, Ca(NO₃)₂, Mg(NO₃)₂ or Zn(NO₃)₂.

12. The process of claim 10, wherein the organic acid is formic acid, acetic acid, oxalic acid, succinic acid, malic acid, tartaric acid, fumaric acid or citric acid.

13. The process of claim 10, wherein the desired ferrosoferric salt concentration is about 10⁻¹⁰ mM.

14. An aqueous ferrosoferric salt solution prepared by a process, comprising:
preparing a solution of trivalent iron salt in water containing a bivalent metal salt or organic acid which supplies counter anion for said ferrosoferric salt;
diluting the aqueous solution with water so as to provide a series of diluted ferrosoferric salt solutions each having a ferrosoferric salt concentration of 10⁻⁴ to 10-20 mM;
measuring the electrical conductivity of each salt solution and selecting only those solutions having an electrical conductivity of more than 3 s/cm;
adding additional trivalent iron salt to each aqueous solution of appropriate electrical conductivity; and then
concentrating the salt solution at a temperature under 100 C to the desired concentration of ferrosoferric salt having the formula: Fe(II)ₘFe(III)ₙY^{-z}_{(2m+3n)/z}
wherein each of m and n is a positive integer, Y is a counter anion for the ferrous and ferric cations and z is the ionic value of counter anion Y.

15. The process of claim 14, wherein the bivalent metal salt is CaCl₂, MgCl₂, ZnCl₂, MgSO₄, Ca(NO₃)₂, Mg(NO₃)₂ or Zn(NO₃)₂.

16. The process of claim 14, wherein the organic acid is formic acid, acetic acid, oxalic acid, succinic acid, malic acid, tartaric acid, fumaric acid or citric acid.

17. A method of restoring animal tissue, comprising:
treating selected animal tissue with the aqueous ferrosoferric salt solution of claim 1.

18. A method of treating the soil, comprising:
treating a soil for crop growth with the aqueous ferrosoferric salt solution of claim 1, thereby improving the growth of the crop under repetitive, seasonal plantings of said crop.

19. An aqueous composition, comprising:
a solution of a ferrosoferric salt of the formula (A):
Fe(II)ₘFe(III)ₙY^{-z}_{(2m+3n)/z}
wherein each of m and n is a positive integer, Y is a counter anion for the ferrous and ferric cations and z is the ionic value of counter anion Y in water, the anti-corrosive activity of the composition being independent of the concentration of the ferrosoferric salt in solution, that is prepared by mixing ferrosoferric salt of formula (A) with distilled water and then diluting the aqueous solution obtained with distilled water.
